(19) United States
(12) Patent Application Publication
(10) Pub. No.: US 2026/0166119 A1
(43) Pub. Date: Jun. 18, 2026

(54) PEPTIDE MIXTURE OF NON-ANIMAL ORIGIN, METHOD FOR PREPARING THE SAME AND USE OF THE SAME FOR PROMOTING REGENERATION OF CELLS

(71) Applicant: MING CHYI BIOTECHNOLOGY LTD., Taipei City (TW)

(72) Inventors: Yi-Heng CHIU, Taichung City (TW); Hung-Chun WANG, Taipei City (TW); Hung-Wen WANG, Taipei City (TW); Han-Ling CHEN, Taipei City (TW)

(21) Appl. No.: 19/174,169

(22) Filed: Apr. 9, 2025

(30) Foreign Application Priority Data
Dec. 18, 2024 (TW) 113149431

(51) Int. Cl. A61K 38/01; A61K 36/48; A61K 36/899

(52) U.S. Cl. CPC A61K 38/011; A61K 36/48; A61K 36/899; A61K 2236/19

(57) ABSTRACT

A peptide mixture is prepared by the steps of: mixing rice and Pisum sativum, followed by conducting a protein extraction treatment, so as to obtain a protein extract; subjecting the protein extract to a hydrolysis treatment with a serine protease at a pH value ranging from 7 to 12, so as to obtain a protein hydrolysate containing a protein precipitate and the peptide mixture; and subjecting the protein hydrolysate to an isolation treatment to remove the protein precipitate, so as to obtain the peptide mixture. Methods for preparing a peptide mixture and promoting regeneration of cells using the peptide mixture are also provided.

### CROSS-REFERENCE TO RELATED APPLICATION

This application claims priority to Taiwanese Invention patent application No. 113149431, filed on Dec. 18, 2024, the entire disclosure of which is incorporated by reference herein.

### FIELD

The present disclosure relates to a peptide mixture and a method for preparing the same. The disclosure also relates to a method for promoting regeneration of cells using the peptide mixture. The cells are selected from the group consisting of skin cells, muscle cells, osteoblasts, chondrocytes, and combinations thereof.

### BACKGROUND

Rice (also known as white rice) is a seed of Oryza sativa, which is a species of the genus Oryza in the Gramineae family. Pisum sativum is an herbaceous plant of the genus Pisum in the Faboideae family, with the pods, seeds, tendrils, and leaves thereof being edible. Both the rice and Pisum sativum are considered essential food crops for humans due to richness in various nutritional content, which includes plant-based proteins, carbohydrates, vitamins, and minerals.

Products derived from rice and Pisum sativum proteins are now widely available on the market, and have been extensively applied in medical and medical aesthetic fields. For instance, the products containing hydrolysates of the rice and Pisum sativum proteins have been found to offer antioxidant and anti-inflammatory properties, as well as being capable of maintaining skin health (such as moisturization, antiaging, and skin firming effects).

In addition, previous studies have subjected the rice and Pisum sativum proteins to a hydrolysis treatment using different enzymes, followed by analyzing changes in components and properties of the resultant protein hydrolysates. For example, Tang, Y. R. et al., (2023), SSRN Electronic Journal, doi: 10.2139/ssrn.4401477 discloses subjecting a pea: rice protein blend to an enzymatic hydrolysis treatment using an enzyme which is selected from the group consisting of pepsin, papain, transglutaminase (TG), a combination of pepsin and TG, and a combination of papain and TG, followed by subjecting the resultant protein hydrolysate to determination of protein content. The protein content data thus obtained show that the protein hydrolysate obtained using the papain has the highest protein content as compared to other enzymes.

In view of the aforesaid, there is still a need to develop an effective protein hydrolysate derived from the rice and Pisum sativum which can be prepared by conducting a hydrolysis treatment using an enzyme, and downstream products of such protein hydrolysate are capable of exhibiting other satisfactory effects.

### SUMMARY

Therefore, in a first aspect, the present disclosure provides a peptide mixture, which can alleviate at least one of the drawbacks of the prior art. The peptide mixture is prepared by the steps of: mixing rice and Pisum sativum, followed by conducting a protein extraction treatment, so as to obtain a protein extract; subjecting the protein extract to a hydrolysis treatment with a serine protease at a pH value ranging from 7 to 12, so as to obtain a protein hydrolysate containing a protein precipitate and the peptide mixture; and subjecting the protein hydrolysate to an isolation treatment to remove the protein precipitate, so as to obtain the peptide mixture. The peptide mixture contains peptides which have an average molecular weight ranging from 500 Da to 1500 Da, and includes, based on the total weight of the peptide mixture as 100 wt %, a total amount of glycine and hydroxyproline that ranges from 10 wt % to 50 wt %, and a total amount of proline and glutamic acid that ranges from 10 wt % to 50 wt %.

In a second aspect, the present disclosure provides a method for promoting regeneration of cells, which can alleviate at least one of the drawbacks of the prior art, and which includes administering to a subject in need thereof a composition including the aforesaid peptide mixture. The cells are selected from the group consisting of skin cells, muscle cells, osteoblasts, chondrocytes, and combinations thereof.

In a third aspect, the present disclosure provides a method for preparing a peptide mixture, which can alleviate at least one of the drawbacks of the prior art, and which includes: mixing rice and Pisum sativum, followed by conducting a protein extraction treatment, so as to obtain a protein extract; subjecting the protein extract to a hydrolysis treatment with a serine protease at a pH value ranging from 7 to 12, so as to obtain a protein hydrolysate containing a protein precipitate and the peptide mixture; and subjecting the protein hydrolysate to an isolation treatment to remove the protein precipitate, so as to obtain the peptide mixture. The peptide mixture contains peptides which have an average molecular weight ranging from 500 Da to 1500 Da, and includes, based on the total weight of the peptide mixture as 100 wt %, a total amount of glycine and hydroxyproline that ranges from 10 wt % to 50 wt %, and a total amount of proline and glutamic acid that ranges from 10 wt % to 50 wt %.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the relative fold change in Hs68 cell growth determined in each group of Example 2, infra, in which the symbol “*” represents p<0.05 (compared with the control group).

FIG. 2 shows the relative fold change in 7F2 cell growth determined in each group as described in section A of Example 3, infra, in which the symbol represents p<0.05 (compared with the control group).

FIG. 3 shows the relative fold change in SW1353 cell growth determined in each group as described in section B of Example 3, infra, in which the symbol “*” represents p<0.05 (compared with the control group).

FIG. 4 shows the relative fold change in C2C12 cell growth determined in each group of Example 4, infra, in which the symbol “*” represents p<0.05 (compared with the control group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a peptide mixture, which is prepared by the steps of: mixing rice and Pisum sativum, followed by conducting a protein extraction treatment, so as to obtain a protein extract; subjecting the protein extract to a hydrolysis treatment with a serine protease at a pH value ranging from 7 to 12, so as to obtain a protein hydrolysate containing a protein precipitate and the peptide mixture; and subjecting the protein hydrolysate to an isolation treatment to remove the protein precipitate, so as to obtain the peptide mixture. The peptide mixture is derived from the rice and Pisum sativum and thus may be of non-animal origin. In addition, the peptide mixture contains peptides which have an average molecular weight ranging from 500 Da to 1500 Da, and includes, based on the total weight of the peptide mixture as 100 wt %, a total amount of glycine and hydroxyproline that ranges from 10 wt % to 50 wt %, and a total amount of proline and glutamic acid that ranges from 10 wt % to 50 wt %.

The present disclosure also provides a method for preparing a peptide mixture, which includes: mixing rice and Pisum sativum, followed by conducting a protein extraction treatment, so as to obtain a protein extract; subjecting the protein extract to a hydrolysis treatment with a serine protease at a pH value ranging from 7 to 12, so as to obtain a protein hydrolysate containing a protein precipitate and the peptide mixture; and subjecting the protein hydrolysate to an isolation treatment to remove the protein precipitate, so as to obtain the peptide mixture. The peptide mixture is derived from the rice and Pisum sativum and thus may be of non-animal origin. In addition, the peptide mixture contains peptides which have an average molecular weight ranging from 500 Da to 1500 Da, and includes, based on the total weight of the peptide mixture as 100 wt %, a total amount of glycine and hydroxyproline that ranges from 10 wt % to 50 wt %, and a total amount of proline and glutamic acid that ranges from 10 wt % to 50 wt %.

In certain embodiments, the peptide mixture may include, based on the total weight of the peptide mixture as 100 wt %, 15 wt % to 50 wt % of the glycine, 0 wt % to 35 wt % of the hydroxyproline, 5 wt % to 20 wt % of the proline, and 5 wt % to 20 wt % of the glutamic acid. In an exemplary embodiment, the peptide mixture includes, based on the total weight of the peptide mixture as 100 wt %, 25.675 wt % of the glycine, 0 wt % of the hydroxyproline, 12.53 wt % of the proline, and 9.056 wt % of the glutamic acid.

As used herein, the term “rice” can be used interchangeably with other terms such as “white rice,” and refers to a seed of Oryza sativa.

As used herein, the term “peptide mixture” can be used interchangeably with other terms such as “hydrolysate of protein extract.”

As used herein, the term “peptide” can be used interchangeably with other terms such as “protein.”

According to the present disclosure, the rice and Pisum sativum used to prepare the peptide mixture may be fresh, unprocessed plant materials, or may be prepared by a treatment selected from the group consisting of a drying treatment, a grinding treatment, a chopping treatment, a comminuting treatment, and combinations thereof. In certain embodiments, before the protein extraction treatment, the rice and Pisum sativum may be subjected to the comminuting treatment using techniques well-known to those skilled in the art.

According to the present disclosure, a weight ratio of the rice to the Pisum sativum may range from 9:1 to 1:9. In an exemplary embodiment, the weight ratio of the rice to the Pisum sativum is 7:3.

According to the present disclosure, the protein extraction treatment may be carried out using techniques well-known to those skilled in the art.

In certain embodiments, the protein extraction treatment includes the step of subjecting a mixture of the rice and Pisum sativum thus obtained to an alkaline extraction treatment using an alkaline solution. A weight ratio of the mixture of the rice and Pisum sativum to the alkaline solution may range from 1:10 to 1:30. In an exemplary embodiment, the weight ratio of the mixture of the rice and Pisum sativum to the alkaline solution is 1:20.

According to the present disclosure, examples of the alkaline solution may include, but are not limited to, sodium hydroxide, sodium carbonate, and sodium bicarbonate. In an exemplary embodiment, the alkaline solution is sodium hydroxide.

According to the present disclosure, the protein extraction treatment may be conducted at a pH value ranging from 9 to 13 for a time period ranging from 1 hour to 5 hours. In an exemplary embodiment, the protein extraction treatment is conducted at the pH value of 11 for the time period of 2 hours.

According to the present disclosure, the serine protease may be obtained as a commercial product, or may be prepared using techniques well-known to those skilled in the art.

According to the present disclosure, the hydrolysis treatment may be carried out using techniques well-known to those skilled in the art.

It should be noted that the operating conditions for the hydrolysis treatment may be adjusted based on factors such as a weight ratio of the protein extract to the serine protease, so as to achieve an optimal hydrolysis result. A selection of these operating conditions for the hydrolysis treatment may be routinely determined by those skilled in the art.

According to the present disclosure, a weight ratio of the protein extract to the serine protease ranges from 1.0:0.005 to 1.0:0.05. In an exemplary embodiment, the weight ratio of the protein extract to the serine protease is 1.0:0.005.

According to the present disclosure, the hydrolysis treatment may be conducted at a temperature ranging from 40° C. to 55° C. for a time period ranging from 30 minutes to 150 minutes. In an exemplary embodiment, the hydrolysis treatment is conducted at the temperature of 45° C. for the time period of 90 minutes.

According to the present disclosure, a pH value of the protein extract is adjusted to 11 before conducting the hydrolysis treatment.

According to the present disclosure, the isolation treatment may be carried out using techniques well-known to those skilled in the art.

In certain embodiments, the isolation treatment may be selected from the group consisting of a centrifugation treatment, a dialysis treatment, a filtration treatment, a precipitation treatment, a salting-out treatment, a column chromatography treatment, and combinations thereof. In an exemplary embodiment, the isolation treatment is the centrifugation treatment.

According to the present disclosure, the centrifugation treatment may be conducted at a temperature ranging from 4° C. to 55° C. and a rotational speed ranging from 500 rpm to 5,000 rpm for a time period ranging from 5 minutes to 30 minutes. In an exemplary embodiment, the centrifugation treatment is conducted at a temperature of 30° C. and a rotational speed of 1,000 rpm for a time period of 20 minutes.

The present disclosure also provides a method for promoting regeneration of cells, which includes administering to a subject in need thereof a composition including the aforesaid peptide mixture. The cells are selected from the group consisting of skin cells, muscle cells, osteoblasts, chondrocytes, and combinations thereof.

As used herein, the term “regeneration” can be used interchangeably with other terms such as “growth,” “proliferation,” and “rejuvenation.”

As used herein, the term “administering” can be used interchangeably with other terms such as “administration,” and means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, the composition may be formulated as a food product using a standard technique well-known to one of ordinary skill in the art. For example, the composition may be formulated in the form of a food additive, which is added to an edible material to prepare a food product for human or animal consumption.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, and dietary supplements.

According to the present disclosure, the composition may be prepared in the form of a pharmaceutical composition. The pharmaceutical composition may be formulated into a dosage form suitable for oral or topical administration using technology well-known to those skilled in the art.

According to the present disclosure, examples of the dosage form suitable for oral administration may include, but are not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well-known to those skilled in the art. Examples of the external preparation may include, but are not limited to, emulsions, gels, ointments, creams, patches, liniments, powders, aerosols, sprays, lotions, serums, pastes, foams, drops, suspensions, salves, and bandages.

According to the present disclosure, the external preparation is prepared by mixing the composition with a base which is well-known to those skilled in the art.

According to the present disclosure, the base may include one or more of the following additives: water, alcohols, glycols, hydrocarbons (e.g., petroleum jelly and white petrolatum), waxes (e.g., paraffin, and yellow waxes), preserving agents, antioxidants, surfactants, absorption enhancers, stabilizing agents, gelling agents (e.g., Carbopol® 974P NF polymers, microcrystalline celluloses, and carboxymethyl cellulose), active agents, humectants, odor absorbers, spices, pH adjusting agents, chelating agents, emulsifiers, occlusive agents, softening agents, thickening agents, solubilizing agents, penetration enhancers, anti-irritants, colorants, propellants, etc. The choice and amount of the aforesaid additives are within the expertise and routine skills of those skilled in the art.

According to the present disclosure, the dose and frequency of administration of the peptide mixture may vary depending on the following factors: the severity of the illness or disorder to be treated, routes of administration, and weight, age, physical condition and response of the subject to be treated. In general, the peptide mixture may be administered in a single dose or in several doses.

According to present disclosure, the composition may be prepared in the form of a cosmeceutical composition. The cosmeceutical composition may be manufactured to a form suitable for skincare or make-up using technology well-known to those skilled in the art.

According to the present disclosure, examples of the form suitable for skincare or make-up may include, but are not limited to, aqueous solutions, aqueous alcohol solutions or oily solutions, oil-in-water type emulsions, water-in-oil type emulsions or composite type emulsions, gels, ointments, creams, masks, patches, packs, liniments, powders, aerosols, sprays, lotions, serums, pastes, foams, dispersions, drops, suspensions, salves, bandages, mousses, sunblock, skin toners, foundation, eyeshadows, makeup removers, soaps, other body cleansing products, etc.

According to present disclosure, the cosmeceutical composition may further include a cosmetically acceptable adjuvant widely employed in the art of cosmetic-manufacturing. For instance, the cosmetically acceptable adjuvant may include one or more of the following agents: solvents, gelling agents, active agents, preservatives, antioxidants, screening agents, chelating agents, surfactants, coloring agents, thickening agents, fillers, spices, and odor absorbers. The choice and amount of the aforesaid agents are within the expertise and routine skills of those skilled in the art.

The disclosure will be further described by way of the following examples. However, it should be understood that the following examples are solely intended for the purpose of illustration and should not be construed as limiting the disclosure in practice.

### EXAMPLES

### General Experimental Materials

### 1. Source and Cultivation of Cell Lines

The type and accession number of each of four cell lines used in the following experiments are shown in Table 1 below. All of the four cell lines were purchased from the Bioresource Collection and Research Center (BCRC) of the Food Industry Research and Development Institute (FIRDI) (No. 331, Shih-Pin Rd., Hsinchu City 300, Taiwan).

TABLE 1

| Type of cell line | Accession number |
|---|---|
| Human skin fibroblast cell line Hs68 | BCRC 60038 |
| Mouse osteoblast cell line 7F2 | BCRC 60453 |
| Human chondrosarcoma cell line SW1353 | BCRC 60548 |
| Mouse myoblast cell line C2C12 | BCRC 60083 |

A respective one of the four cell lines was grown in a Petri dish containing a respective one of the media as shown in Table 2 below, followed by cultivating in an incubator at 37° C. with 5% CO₂. Subsequently, medium change was performed every two to three days. When the Hs68 cells, the 7F2 cells, and the SW1353 cells reached approximately 80% to 90% confluence, and the C2C12 cells reached approximately 70% to 80% confluence, cell passage was performed.

TABLE 2

| Cell line | Type of medium and recipe therein |
|---|---|
| Hs68 | Dulbecco's modified Eagle's medium (DMEM, |
| cells | Manufacturer: Thermo Fisher Scientific, Inc.) |
| | supplemented with 10% fetal bovine serum (FBS) |
| 7F2 cells | Minimal essential medium Eagle-alpha modification (α- |
| | MEM, Manufacturer: Sigma-Aldrich) supplemented with |
| | 10% FBS, 2 mM of L-glutamine, 1.5 g/L of sodium |
| | bicarbonate, and 1 mM of sodium pyruvate |
| SW1353 | DMEM (Manufacturer: Thermo Fisher Scientific, Inc.) |
| cells | supplemented with 10% FBS |
| C2C12 | DMEM (Manufacturer: Thermo Fisher Scientific, Inc.) |
| cells | supplemented with 10% FBS, 4 mM of L-glutamine, 1.5 |
| | g/L of sodium bicarbonate, and 4.5 g/L of glucose |

### 2. Type and Source of Proteases

The type and source of each of the proteases used in the following experiments are shown in Table 3 below.

TABLE 3

| Type of protease | Source | Cat. no. |
|---|---|---|
| Serine protease | BIO-CAT, Inc. | MCF00130094 |
| Papain | Sigma-Aldrich | 10108014001 |
| Pepsin | Merck, Ltd. | 516360 |

### General Procedures:

### 1. Statistical Analysis

All the experiments described below were performed in triplicates. The experimental data of all the groups are expressed as mean±standard deviation (SD), and were analyzed using SPSS Statistics software, so as to evaluate the differences between the groups. Statistical significance is indicated by p<0.05.

### Example 1. Preparation of Peptide Mixture

First, a rice powder (Manufacturer: INGRETRA of Ming Chyi Biotechnology Ltd.; Cat. no.: MCBR850) and a Pisum sativum powder (Manufacturer: INGRETRA of Ming Chyi Biotechnology Ltd.; Cat. no.: MCBP850) were mixed at a weight ratio of 7:3, followed by conducting a homogenization treatment using a high shear homogenizer (Manufacturer: ELE company; Model no.: ELR420/3) for a time period of 10 minutes, so as to obtain a homogeneous mixture.

Next, the homogeneous mixture was subjected to a protein extraction treatment that included the following steps: adding the homogeneous mixture into a sodium hydroxide solution (with a pH value of 11) at a weight ratio of 1:20, followed by mixing well, so as to obtain a mixture; and subjecting the mixture to a extraction treatment at a temperature of 55° C. for a time period of 2 hours, so as to obtain a protein extract.

Thereafter, a pH value of the protein extract is adjusted to 11 using a sodium hydroxide solution, so as to obtain an adjusted protein extract. The adjusted protein extract was then mixed with a serine protease at a weight ratio of 1.0:0.005, followed by conducting a hydrolysis treatment at a temperature of 45° C. for a time period of 90 minutes, so as to obtain a protein hydrolysate containing a macromolecular protein precipitate and a peptide mixture. Afterwards, the protein hydrolysate was subjected to a centrifugation treatment (serving as an isolation treatment) at a temperature of 30° C. and a rotational speed of 1,000 rpm for a time period of 20 minutes to remove the macromolecular protein precipitate, so as to collect the resultant supernatant containing the peptide mixture. After that, SGS Taiwan Ltd. was commissioned by the Applicant to determine molecular weight of the supernatant containing the peptide mixture. The result showed that the peptide mixture contained peptides having an average molecular weight ranging from 500 Da to 1500 Da.

Afterwards, the supernatant containing the peptide mixture was subjected to a spray-drying treatment to remove moisture while increasing a concentration of the peptide mixture therein, thereby obtaining a powder of the peptide mixture (abbreviated as peptide mixture of the present disclosure).

Next, SGS Taiwan Ltd. was commissioned by the Applicant to analyze amino acid components of the peptide mixture of the present disclosure. Briefly, 100 g of the powder of the peptide mixture was subjected to an acid hydrolysis treatment using hydrochloric acid containing phenol for a time period ranging from 20 hours to 24 hours so as to obtain a test sample, and then the test sample was subjected to high performance liquid chromatography (HPLC) analysis using post-column derivatization. The result is shown in Table 4 below.

TABLE 4

| Amino acid | Content (wt %, based on total weight of |
|---|---|
| component | peptide mixture of the present disclosure) |
| Glycine | 25.675 |
| Proline | 12.530 |
| Hydroxyproline | 0 |
| Glutamic acid | 9.056 |
| Alanine | 3.103 |
| Arginine | 5.133 |
| Aspartic acid | 5.289 |
| Leucine | 3.999 |
| Serine | 2.717 |
| Isoleucine | 2.250 |
| Valine | 2.791 |
| Threonine | 1.809 |
| Phenylalanine | 2.455 |
| Histidine | 1.351 |
| Lysine | 2.547 |
| Methionine | 0.935 |
| Cysteine | 0.604 |
| Tyrosine | 2.284 |

In addition, comparative hydrolysates 1 and 2 were prepared for comparison purpose. Briefly, a protein hydrolysate was not subjected to the isolation treatment but was instead directly subjected to the spray-drying treatment, so as to obtain a powder of the protein hydrolysate (abbreviated as comparative hydrolysate 1).

The procedures for preparing the comparative hydrolysate 2 were similar to those of the comparative hydrolysate 1, except that the sodium hydroxide solution (with a pH value of 11) used in the protein extraction treatment and pH adjustment of the protein extract was replaced by phosphate-buffered saline (PBS, with a pH value of 5.8), and the serine protease was replaced by an enzyme blend prepared by mixing papain and pepsin at a weight ratio of 1:1.

### Example 2. Evaluation of the Effect of Peptide Mixture of the Present Disclosure on Promoting Regeneration of Skin Cells

### Experimental Materials

### 1. Preparation of Test Solutions

Briefly, a suitable amount of a respective one of the peptide mixture of the present disclosure and the comparative hydrolysates 1 and 2 prepared in Example 1 was dissolved in a suitable amount of phosphate-buffered saline (PBS), so as to obtain a test solution of the respective one of the peptide mixture of the present disclosure and the comparative hydrolysates 1 and 2.

### Experimental Procedures

First, the Hs68 cells prepared in section 1 of “General Experimental Materials” were divided into 4 groups, including a control group, an experimental group, and two comparative groups (i.e., comparative groups 1 and 2). The Hs68 cells of each group was seeded at a concentration of 1×10⁴ cells/well into a respective well of a 96-well culture plate containing 200 μL of DMEM, followed by cultivation in an incubator (37° C., 5% CO₂) for 24 hours.

Next, the culture medium in each group was removed to be replaced with a fresh culture medium. Subsequently, the Hs68 cells of each of the experimental group and the comparative groups 1 and 2 were treated with a suitable amount of a corresponding test solution prepared in section 1 of “Experimental Materials” of this example as shown in Table 5 below. In addition, the Hs68 cells of the control group received no treatment.

TABLE 5

| | Test solution |
|---|---|
| Group | (final concentration: 2% (w/w)) |
| Control group | — |
| Experimental group | Test solution of peptide mixture of the |
| | present disclosure |
| Comparative group 1 | Test solution of comparative hydrolysate 1 |
| Comparative group 2 | Test solution of comparative hydrolysate 2 |

Thereafter, each group was cultivated in an incubator (37° C., 5% CO₂) for 24 hours, followed by conducting cell counting using a cell counting chamber, so as to determine cell count of each group.

The relative fold change in Hs68 cell growth for each group was calculated by substituting the thus determined cell count into the following Equation (1):

$$A = B / C \qquad ( 1 )$$

where A=relative fold change in Hs68 cell growth
B=cell count determined in each group
C=cell count determined in the control group

The data thus obtained were analyzed according to the procedures as described in section 1 of “General Procedures.”

### Results:

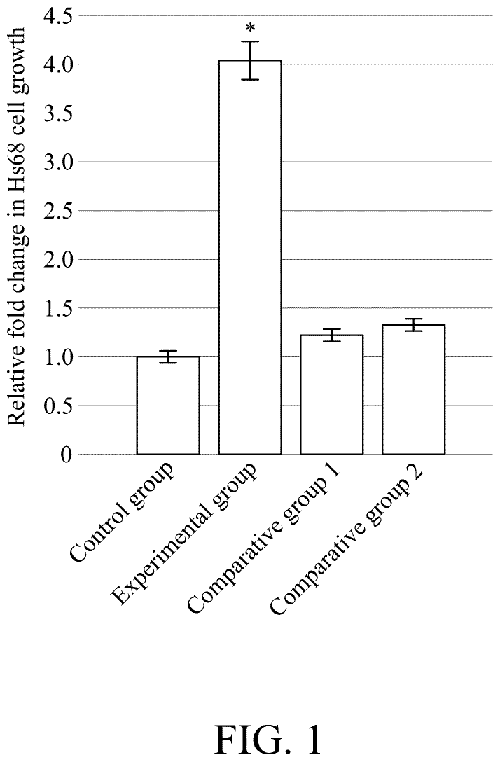

FIG. 1 shows the relative fold change in Hs68 cell growth determined in each group. As shown in FIG. 1, compared with the control group, the relative fold change in Hs68 cell growth determined in each of the comparative groups 1 and 2 showed no significant difference, whereas the relative fold change in Hs68 cell growth determined in the experimental group showed a significant increase, i.e., approximately 4-fold increase. These results demonstrate that the peptide mixture of the present disclosure can effectively promote regeneration of the Hs68 cells, and such effect is significantly better than that of the comparative hydrolysates 1 and 2.

### Example 3. Evaluation of the Effect of Peptide Mixture of the Present Disclosure on Promoting Regeneration of Bone Cells

A. 7F2 Cells Treated with Peptide Mixture of the Present Disclosure

### Experimental Procedures

First, the 7F2 cells prepared in section 1 of “General Experimental Materials” were divided into 4 groups, including a control group, an experimental group, and two comparative groups (i.e., comparative groups 1 and 2). The 7F2 cells of each group was seeded at a concentration of 1×10⁴ cells/well into a respective well of a 96-well culture plate containing 200 μL of α-MEM, followed by cultivation in an incubator (37° C., 5% CO₂) for 24 hours.

Next, the 7F2 cells of each of the experimental group and the comparative groups 1 and 2 were treated with a suitable amount of a corresponding test solution prepared in section 1 of “Experimental Materials” of Example 2 as shown in Table 6 below, while the 7F2 cells of the control group received no treatment, and all subsequent steps, including determination of cell count and calculation of the relative fold change in 7F2 cell growth, were performed according to the procedures as described in Example 2.

TABLE 6

| | Test solution |
|---|---|
| Group | (final concentration: 2% (w/w)) |
| Control group | — |
| Experimental group | Test solution of peptide mixture of the |
| | present disclosure |
| Comparative group 1 | Test solution of comparative hydrolysate 1 |
| Comparative group 2 | Test solution of comparative hydrolysate 2 |

The data thus obtained were analyzed according to the procedures as described in section 1 of “General Procedures.”

### Results:

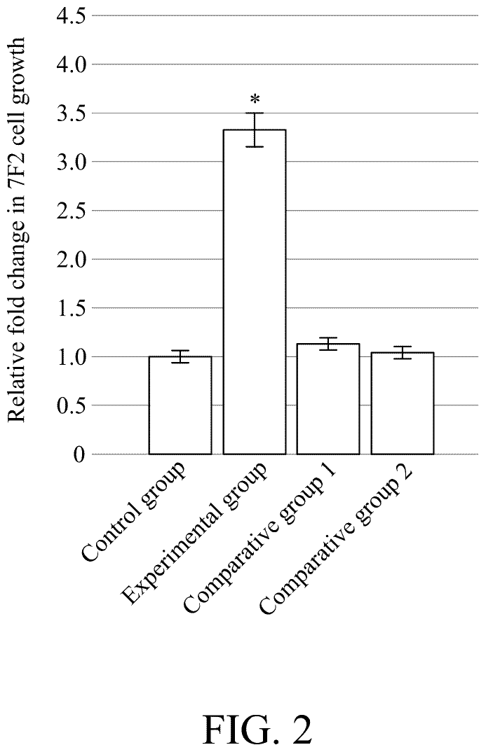

FIG. 2 shows the relative fold change in 7F2 cell growth determined in each group. As shown in FIG. 2, compared with the control group, the relative fold change in 7F2 cell growth determined in each of the comparative groups 1 and 2 showed no significant difference, whereas the relative fold change in 7F2 cell growth determined in the experimental group showed a significant increase, i.e., more than 3-fold increase. These results demonstrate that the peptide mixture of the present disclosure can effectively promote regeneration of the 7F2 cells, and such effect is significantly better than that of the comparative hydrolysates 1 and 2.

B. SW1353 Cells Treated with Peptide Mixture of the Present Disclosure

### Experimental Procedures

First, the SW1353 cells prepared in section 1 of “General Experimental Materials” were divided into 4 groups, including a control group, an experimental group, and two comparative groups (i.e., comparative groups 1 and 2). The SW1353 cells of each group was seeded at a concentration of 1×10⁴ cells/well into a respective well of a 96-well culture plate containing 200 μL of DMEM, followed by cultivation in an incubator (37° C., 5% CO₂) for 24 hours.

Nest, the SW1353 cells of each of the experimental group and the comparative groups 1 and 2 were treated with a suitable amount of a corresponding test solution prepared in section 1 of “Experimental Materials” of Example 2 as shown in Table 7 below, while the SW1353 cells of the control group received no treatment, and all subsequent steps, including determination of cell count and calculation of the relative fold change in SW1353 cell growth, were performed according to the procedures as described in Example 2.

TABLE 7

| | Test solution |
|---|---|
| Group | (final concentration: 2% (w/w)) |
| Control group | — |
| Experimental group | Test solution of peptide mixture of the |
| | present disclosure |
| Comparative group 1 | Test solution of comparative hydrolysate 1 |
| Comparative group 2 | Test solution of comparative hydrolysate 2 |

The data thus obtained were analyzed according to the procedures as described in section 1 of “General Procedures.”

### Results:

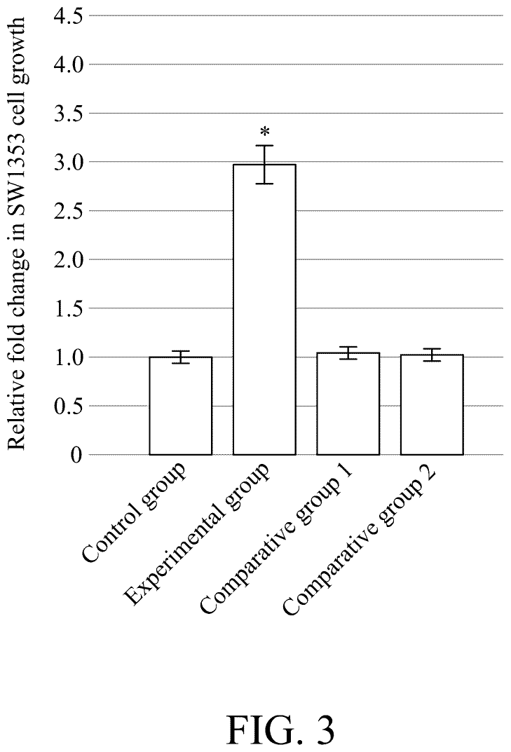

FIG. 3 shows the relative fold change in SW1353 cell growth determined in each group. As shown in FIG. 3, compared with the control group, the relative fold change in SW1353 cell growth determined in each of the comparative groups 1 and 2 showed no significant difference, whereas the relative fold change in SW1353 cell growth determined in the experimental group showed a significant increase, i.e., approximately 3-fold increase. These results demonstrate that the peptide mixture of the present disclosure can effectively promote regeneration of the SW1353 cells, and such effect is significantly better than that of the comparative hydrolysates 1 and 2.

### Example 4. Evaluation of the Effect of Peptide Mixture of the Present Disclosure on Promoting Regeneration of Muscle Cells

### Experimental Procedures

First, the C2C12 cells prepared in section 1 of “General Experimental Materials” were divided into 4 groups, including a control group, an experimental group, and two comparative groups (i.e., comparative groups 1 and 2). The C2C12 cells of each group was seeded at a concentration of 1×10⁴ cells/well into a respective well of a 96-well culture plate containing 200 μL of DMEM, followed by cultivation in an incubator (37° C., 5% CO₂) for 24 hours.

Next, the C2C12 cells of each of the experimental group and the comparative groups 1 and 2 were treated with a suitable amount of a corresponding test solution prepared in section 1 of “Experimental Materials” of Example 2 as shown in Table 8 below, while the C2C12 cells of the control group received no treatment, and all subsequent steps, including determination of cell count and calculation of the relative fold change in C2C12 cell growth, were performed according to the procedures as described in Example 2.

TABLE 8

| | Test solution |
|---|---|
| Group | (final concentration: 2% (w/w)) |
| Control group | — |
| Experimental group | Test solution of peptide mixture of the |
| | present disclosure |
| Comparative group 1 | Test solution of comparative hydrolysate 1 |
| Comparative group 2 | Test solution of comparative hydrolysate 2 |

The data thus obtained were analyzed according to the procedures as described in section 1 of “General Procedures.”

### Results:

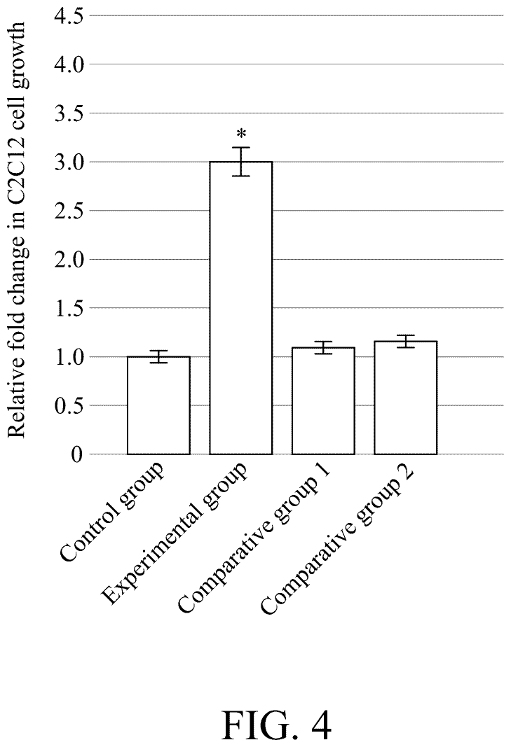

FIG. 4 shows the relative fold change in C2C12 cell growth determined in each group. As shown in FIG. 4, compared with the control group, the relative fold change in C2C12 cell growth determined in each of the comparative groups 1 and 2 showed no significant difference, whereas the relative fold change in C2C12 cell growth determined in the experimental group showed a significant increase, i.e., approximately 3-fold increase. These results demonstrate that the peptide mixture of the present disclosure can effectively promote regeneration of the C2C12 cells, and such effect is significantly better than that of the comparative hydrolysates 1 and 2.

Summarizing the above test results, it is clear that by virtue of subjecting the protein extract derived from the rice and Pisum sativum to the hydrolysis treatment with the serine protease, followed by conducting the isolation treatment, the thus obtained peptide mixture, which contains peptides having an average molecular weight ranging from 500 Da to 1500 Da, is capable of effectively promoting regeneration of skin cells, bone cells (e.g., osteoblasts and chondrocytes), and muscle cells, and such effect is significantly better than that of the comparative hydrolysate 2, which is prepared by subjecting the protein extract derived from the rice and Pisum sativum to the hydrolysis treatment with the enzyme blend of papain and pepsin. Therefore, the peptide mixture of the present disclosure has a high potential to be used in the preparation of a product for promoting regeneration of skin cells, bone cells, and muscle cells.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A peptide mixture, which is prepared by the steps of:
mixing rice and Pisum sativum, followed by conducting a protein extraction treatment, so as to obtain a protein extract;
subjecting the protein extract to a hydrolysis treatment with a serine protease at a pH value ranging from 7 to 12, so as to obtain a protein hydrolysate containing a protein precipitate and the peptide mixture; and
subjecting the protein hydrolysate to an isolation treatment to remove the protein precipitate, so as to obtain the peptide mixture;
wherein the peptide mixture contains peptides which have an average molecular weight ranging from 500 Da to 1500 Da, and includes, based on the total weight of the peptide mixture as 100 wt %,
a total amount of glycine and hydroxyproline that ranges from 10 wt % to 50 wt %, and
a total amount of proline and glutamic acid that ranges from 10 wt % to 50 wt %.

2. The peptide mixture as claimed in claim 1, wherein a weight ratio of the rice to the Pisum sativum ranges from 9:1 to 1:9.

3. A method for promoting regeneration of cells, comprising administering to a subject in need thereof a composition including the peptide mixture as claimed in claim 1,
wherein the cells are selected from the group consisting of skin cells, muscle cells, osteoblasts, chondrocytes, and combinations thereof.

4. The method as claimed in claim 3, wherein the composition is selected from the group consisting of a food product, a pharmaceutical composition, and a cosmeceutical composition.

5. The method as claimed in claim 4, wherein the pharmaceutical composition is administered by a route selected from the group consisting of oral administration and topical administration.

6. A method for preparing a peptide mixture, comprising:
mixing rice and Pisum sativum, followed by conducting a protein extraction treatment, so as to obtain a protein extract;
subjecting the protein extract to a hydrolysis treatment with a serine protease at a pH value ranging from 7 to 12, so as to obtain a protein hydrolysate containing a protein precipitate and the peptide mixture; and
subjecting the protein hydrolysate to an isolation treatment to remove the protein precipitate, so as to obtain the peptide mixture;
wherein the peptide mixture contains peptides which have an average molecular weight ranging from 500 Da to 1500 Da, and includes, based on the total weight of the peptide mixture as 100 wt %,
a total amount of glycine and hydroxyproline that ranges from 10 wt % to 50 wt %, and
a total amount of proline and glutamic acid that ranges from 10 wt % to 50 wt %.

7. The method as claimed in claim 6, wherein a weight ratio of the rice to the Pisum sativum ranges from 9:1 to 1:9.
